(19) United States
(12) Patent Application Publication
(10) Pub. No.: US 2004/0038240 A1
(43) Pub. Date: Feb. 26, 2004

(54) Method and materials for assessing cancer aggressiveness

(76) Inventors: Akhouri Sinha, Minneapolis, MN (US); Michael J. Wilson, St. Paul, MN (US); Barry J. Quast, Minneapolis, MN (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/332,628

(22) PCT Filed: Jul. 13, 2001
(86) PCT No.: PCT/US01/22072

(51) Int. Cl. C12Q 1/68; C12P 19/34

(52) U.S. Cl. 435/6; 435/91.2

(57) ABSTRACT

The invention involves the predicting the aggressiveness of cancer. Specifically, the invention provides methods and materials for evaluating the ratio of CB to stefin A such that the aggressiveness of cancer can be predicted.

### STATEMENT AS TO FEDERALLY SPONSORED RESEARCH

Funding for the work described herein was provided in part by the federal government, which may have certain rights in the invention.

### BACKGROUND

1. Technical Field

The invention relates to methods and materials involved in the prediction of cancer aggressiveness. Specifically, the invention provides methods and materials for measuring enzyme/inhibitor ratios such that aggressive cancers can be identified.

2. Background Information

Cancers can be graded according to structural features that reflect the degree of differentiation. The better differentiated a tumor is, the more it resembles its tissue of origin. Poorly-differentiated tumors have less resemblance to their tissues of origin, and are generally more malignant than well differentiated tumors. Tumor grading, therefore, can provide an indication of a tumor's degree of malignancy.

At the time of initial diagnosis of human prostate cancer (CaP), neoplastic cells may have already spread to extraprostatic organs in about 50% of patients while in others they may be confined to the prostate gland. In spite of several advances in diagnosis and treatment of prostate cancer during the past 50 years, prediction of the clinical course of this cancer in patients remains unsatisfactory. To improve diagnosis and treatment of prostate cancers, Gleason proposed a prostate tumor grading system based upon the histological analysis of a very large set of prostate cancer cases correlated to patient survival and mortality. The Gleason grading system classifies prostate tumors into nine distinct histologic scores ranging from 2 to 10. Histologic scores can be grouped into three levels of differentiation representing well (2 to 4), moderately (5 to 7), and poorly (8 to 10) differentiated tumors. About 75% of prostate cancer cases fall into the moderately-differentiated tumor category, and are sometimes called intermediate grade tumors.

In general, the lower the Gleason histologic score number, the better the prognosis of the disease. There are numerous individuals, however, who do not follow the course predicted by tumor grading. A variety of prognostic factors such as pathological grades and/or histologic scores, surgical margins of prostatic tumors, serum total prostate specific antigen (PSA), free PSA, complexed PSA levels and/or their ratios, and clinical stages have been used in making clinical decisions for diagnosis and treatment of prostate cancer patients. These factors, however, are unable to predict the clinical outcomes in many cases of prostate cancer patients. This is because the factors do not distinguish biologically aggressive prostate cancers, for example, within a single Gleason histologic score category that may contain both aggressive and less aggressive forms of cancers. Thus, there is an urgent need to define selected parameters involved in tumor progression leading to differential diagnosis and treatment of prostate cancer in patients.

Hydrolytic enzymes and their inhibitors are involved in degradation of the extracellular matrix, an event thought to be necessary for tumor progression. Cathepsin B (CB), a lysosomal cysteine protease, is involved in degradation of extracellular matrix proteins. The function of CB is regulated in part by endogenous cysteine protease inhibitors (CPIs) (such as the cystatins or stefins, and specifically stefin A).

### SUMMARY

The invention is based on the discovery that enzyme/inhibitor ratios correlate with the aggressiveness of a cancer. Hydrolytic enzymes that can degrade or remodel the extracellular matrix are regulated by endogenous inhibitors. When enzyme levels surpass inhibitor levels in the vicinity of a tumor, the increase in extracellular matrix degradation and remodeling can promote invasion of the tissues surrounding the tumor, as well as metastasis to other organs. Measuring the levels of enzyme and inhibitor biomolecules in a primary organ cancer sample, and using those levels to calculate an enzyme/inhibitor ratio, can help predict whether that cancer is aggressive. Identifying aggressive cancers at an early stage can help a physician to properly diagnose and treat a cancer patient. Typically, a properly diagnosed and treated cancer patient can experience an improvement in general health and survival.

For example, the ratio of CB to stefin A can be determined in a sample from a patient. If the ratio is such that more CB than stefin A exists in that sample, then the corresponding cancer can be considered aggressive. Either RNA or polypeptide levels of CB and stefin A can be assessed to determine the aggressiveness of a cancer. Typically, if the ratio of CB to stefin A is greater than about 1.0 then the cancer can be considered aggressive, while a ratio less than about 1.0 indicates that the cancer is less aggressive. Knowing the aggressiveness of a particular cancer can greatly aid in treatment and management of cancer patients.

In general, the invention provides a method for determining the aggressiveness of a cancer in a mammal. The method includes determining the ratio of a cathepsin mRNA to a stefin mRNA in the mammal, and determining the aggressiveness of that cancer based on that ratio. A cancer can be considered to be aggressive if the ratio is greater than 1.0, 3.0, or 5.0. The cancer can be a prostate, breast, skin, brain, colon, lung, ovary, or bladder cancer. The ratio can be determined in tissue from the cancer. The ratio can be determined by RT-PCR or in situ hybridization. The cathepsin mRNA being measured can be a CB mRNA. The stefin mRNA being measured can be a stefin A mRNA.

The term “greater than” means that a particular ratio is statistically significantly higher than a particular value at p<0.05. For example, a ratio greater than 1.0 can be any ratio that is statistically significantly greater than 1.0 at p<0.05.

In another aspect, the invention provides a method for determining the aggressiveness of a cancer in a mammal by determining the ratio of cathepsin polypeptide to stefin polypeptide, and determining the aggressiveness of that cancer based on that ratio. Again, the cancer can be considered aggressive if the ratio is greater than 1.0, 3.0, or 5.0. The cancer can be a prostate, breast, skin, brain, colon, lung, ovary, or bladder cancer. The ratio can be determined in tissue from the cancer. The ratio can be determined by ELISA, confocal microscopy, immunofluorescence, or immunohistochemistry. The cathepsin polypeptide being measured can be a CB polypeptide. The stefin polypeptide being measured can be a stefin A polypeptide.

Another embodiment of the invention features a method for determining whether or not a cancer in a mammal is metastatic. The method includes determining the ratio of a cathepsin polypeptide to a stefin polypeptide in the mammal, and determining whether or not that cancer is metastatic based on that ratio. The cancer can be considered metastatic if the ratio is greater than 2.5, 3.0, 5.0, or 7.0. The cancer can be a prostate, breast, skin, brain, colon, lung, ovary, or bladder cancer. The ratio can be determined in tissue from the cancer. The ratio can be determined by ELISA, confocal microscopy, immunofluorescence, or immunohistochemistry. The cathepsin polypeptide being measured can be a CB polypeptide. The stefin polypeptide being measured can be a stefin A polypeptide.

The invention also features a kit comprising a first oligonucleotide primer pair and a second oligonucleotide primer pair. The first oligonucleotide primer pair can amplify a CB nucleic acid and the second oligonucleotide primer pair can amplify a stefin A nucleic acid.

In another embodiment, the invention features a kit comprising a first oligonucleotide probe and a second oligonucleotide probe. The first oligonucleotide probe can hybridize under moderate to high stringency conditions to a CB nucleic acid and the second oligonucleotide probe can hybridize under moderate to high stringency conditions to a stefin A nucleic acid. One or both probes can be labeled.

Another embodiment features a kit comprising a first antibody and a second antibody. The first antibody can have specific binding affinity for a CB polypeptide and the second antibody can have specific binding affinity for a stefin A polypeptide.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1A is a bar graph depicting the distribution of CB>stefin A (mRNA) by Gleason grade in normal prostate (N), benign prostatic hyperplasia (BPH), and neoplastic human prostates having Gleason histological scores of 3, 5, 6, and 9 and 10. FIG. 1B is a bar graph depicting the distribution of CB<stefin A (mRNA) by Gleason grade in neoplastic human prostates having Gleason histological scores of 3, 6, and 7. FIG. 1C is a bar graph depicting the distribution of CB=stefin A (mRNA) by Gleason grade in neoplastic human prostates having Gleason histological scores of 3 and 4, 5, 6, and 7.

FIG. 2 is a bar graph depicting CB/stefin A polypeptide ratios in sections of BPH and Gleason score 6 prostate samples. Bars represent the standard error of the mean. Student's t-test, p<0.05.

FIG. 3a is a bar graph depicting CB activity in homogenates (Homog) and membrane/endosomal (M/E) fractions of hyperplastic (BPH) and neoplastic (CAP) human prostate samples. Data are expressed as the mean nmoles/min·mg protein±standard deviation. FIG. 3b is a bar graph depicting the activity of heat stable cysteine protease inhibitors in hyperplastic (BPH) and neoplastic (CAP) human prostate. Data are expressed as the mean units/mg protein±standard deviation.

FIG. 4a is a bar graph depicting the average CB/stefin A polypeptide ratios in primary prostate cancer samples as related to metastasis-positive lymph node, metastasis-negative lymph node, and BPH samples. FIG. 4b is a bar graph depicting the average CB/stefin A polypeptide ratios in primary prostate cancer samples as related to CB>stefin A, CB<stefin A, CB=stefin A, and BPH samples. Data are presented as the mean±SEM.

### DETAILED DESCRIPTION

In general, the invention provides methods for determining the aggressiveness of a cancer by measuring an enzyme/inhibitor ratio. Without being bound by a particular mechanism, enzymes that can degrade or remodel the extracellular matrix are regulated by endogenous inhibitors. When enzyme levels surpass inhibitor levels in the vicinity of a tumor, the increase in extracellular matrix degradation and remodeling can promote invasion of the tissues surrounding the tumor, as well as metastasis to other organs. Measuring the levels of enzyme and inhibitor biomolecules in a cancer sample, and using those levels to calculate an enzyme/inhibitor ratio, can help predict whether that cancer is aggressive. Identifying aggressive cancers at an early stage can help a physician to properly diagnose and treat a cancer patient. Typically, a properly diagnosed and treated cancer patient can experience an improvement in general health and survival.

The term “aggressive” as used herein refers to the invasive and metastatic activity of a cancer. For example, an aggressive prostate cancer is more invasive and metastatic than a less aggressive prostate cancer. Aggressive cancers can produce adverse changes in a mammal's overall health to a greater extent than if that cancer were not aggressive. A mammal with an aggressive prostate cancer can, for example, experience bladder obstruction problems (e.g., slow urinary stream, nocturia, or dysuria) to a greater extent than if that prostate cancer were not aggressive. Other adverse changes in overall health include, without limitation, pain associated with metastasis, edema, enlargement of organs such as lymph nodes, lungs, and liver, pathological bone fractures, loss of appetite, mineral and vitamin deficiencies, increased risk of infection, and depression. Aggressive cancers can increase mortality to a greater extent than less aggressive cancers. Any type of cancer can exhibit various degrees of aggressiveness, including, without limitation, solid tissue cancers such as prostate, breast, skin (e.g., melanoma), brain, colon, lung, ovary, and bladder.

The ratio of an enzyme to its inhibitor can be used to identify aggressive cancers, as well as to determine the degree of aggressiveness of a cancer. An enzyme/inhibitor ratio can be calculated from the biomolecule levels of a particular enzyme and its inhibitor measured in a cancer sample or in other biological samples from a cancer patient. The term “biomolecule” as used herein refers to RNAs and polypeptides. For example, an enzyme mRNA level and an inhibitor mRNA level can be used to calculate an enzyme/inhibitor ratio. Biomolecule levels of enzymes and inhibitors typically involved in cancer invasion and metastasis can be measured. Such enzymes include, without limitation, metalloproteases (MMPs), serine proteases such as plasminogen activator, and cysteine proteases such as the cathepsins. Non-limiting examples of suitable inhibitors include tissue inhibitors of metalloproteases (TIMPs), serine protease inhibitors (SPIs) such as the serpins, and cysteine protease inhibitors (CPIs) such as the stefins (also known as the cystatins). mRNA levels of cathepsin B (CB) and its inhibitor stefin A can, for example, be measured in a prostate cancer sample from a patient. The measured mRNA levels can be used to calculate a ratio of CB to stefin A (i.e., enzyme to inhibitor). If that CB/stefin A ratio is higher than a corresponding control ratio, then the prostate cancer in that patient can be classified as aggressive. Suitable control ratios include, but are not limited to, an average ratio of several patients without prostate cancer, or an average ratio of several patients with benign prostatic hyperplasia (BPH). Alternatively, if an enzyme/inhibitor ratio for a particular cancer has been characterized such that a standard control ratio is known, comparing a ratio to the standard for that particular cancer can identify an aggressive cancer. For example, a CB/stefin A ratio of 1.0 or greater indicates that a prostate cancer is aggressive.

As the enzyme/inhibitor ratio can be used to classify a cancer as aggressive, so can the enzyme/inhibitor ratio be used to determine the degree of aggressiveness of a cancer. The higher the ratio, the greater the degree of aggressiveness of the corresponding cancer. When comparing two CB/stefin A ratios, for example, if the first ratio is higher than the second ratio then the prostate cancer corresponding to the first ratio likely is more aggressive than the prostate cancer corresponding to the second ratio. Enzyme/inhibitor ratios from cancer samples taken at different times from the same cancer (i.e., same patient) also can be compared. Such a comparison can determine whether the aggressiveness of that cancer has changed with time. CB and stefin A polypeptide levels can, for example, be measured in a prostate cancer sample X and in a prostate cancer sample Y taken 9 months later from the same patient. If the calculated CB/stefin A ratio of sample Y is less than that of sample X, then the aggressiveness of that prostate cancer in that patient has decreased over those 9 months. Ratios corresponding to different patients having the same type of cancer can be compared to each other, providing a measurement of the relative degree of aggressiveness of those cancers. For example, if a first prostate cancer patient has a CB/stefin A ratio that is higher than that of a second prostate cancer patient, then the first prostate cancer patient likely has a more aggressive prostate cancer than that of the second prostate cancer patient. Enzyme/inhibitor ratios can be used in conjunction with current cancer aggressiveness scoring systems. CB/stefin A ratios can, for example, be used to identify aggressive and less aggressive prostate cancers that have the same Gleason histologic score. Prostate cancers with various degrees of aggressiveness can occur within any individual Gleason score category even though prostate cancers with higher Gleason scores are considered more aggressive than prostate cancers with lower scores. Knowing to what extent aggressiveness varies among cancers with similar Gleason scores can lead to more accurate diagnosis of patients with aggressive cancers, and improvements in their individual treatments leading to increased survival.

Enzyme/inhibitor ratios can be particularly useful in evaluating the metastatic status of a cancer. The term “metastatic” as used herein refers to the status of a cancer that has metastasized or, absent therapeutic intervention, will metastasize. Typically, an aggressive cancer metastasizes to a particular organ, e.g., an aggressive prostate cancer typically metastasizes to lymph nodes (e.g., pelvic lymph nodes). An enzyme/inhibitor ratio measured in a sample from the primary cancer can be used to assess the probability that an aggressive cancer has or will metastasize to that particular organ. CB and stefin A polypeptide levels can, for example, be measured in a sample from a prostate cancer patient and used to calculate a CB/stefin A ratio. If the CB/stefin A ratio is higher than a corresponding control ratio, then that prostate cancer has a high probability of being metastatic in that patient. Suitable control ratios include, but are not limited to, an average ratio of several prostate cancer patients without lymph node metastasis, or an average ratio of several patients with BPH. Again, if an enzyme/inhibitor ratio for a particular cancer has been characterized such that a standard control ratio is known, the metastatic status of that particular cancer can be determined by comparing a ratio to the standard for that particular cancer. For example, a CB/stefin A ratio of 3.0 or greater indicates that an aggressive prostate cancer is metastatic.

It is understood that ratios of the same enzyme/inhibitor combination are compared when determining the degree of aggressiveness of a cancer. Further, ratios corresponding to the same type of cancer are compared. For example, when measuring a CB/stefin A ratio to determine the degree of aggressiveness of a prostate cancer, that CB/stefin A ratio is compared to other CB/stefin A ratios from prostate cancer samples, such as control CB/stefin A ratios. Typically, levels of the same type of biomolecule are used when calculating a ratio (i.e., enzyme mRNA to inhibitor mRNA or enzyme polypeptide to inhibitor polypeptide). In some circumstances, however, it may be useful to use different types of biomolecules to calculate a ratio (i.e., enzyme mRNA to inhibitor polypeptide or enzyme polypeptide to inhibitor mRNA).

Any sample can be used when measuring the levels of enzyme and inhibitor biomolecules. Such samples include, without limitation, blood (e.g. venous prostate blood), serum, tissue biopsies, surgical waste, isolated cells, and whole organs. When measuring biomolecule levels for determining the aggressiveness of a prostate cancer, suitable samples can also include tissue samples from transurethral resection of the prostate (TURP). A sample can be manipulated prior to measuring the levels of enzyme and inhibitor biomolecules. For example, prostatectomy surgical waste from a prostate cancer patient can be treated such that total mRNA is obtained. In another example, a prostate cancer biopsy can be frozen, embedded, sectioned, and stained to identify cancerous regions.

Any method can be used to measure the levels of enzyme and inhibitor biomolecules in a sample. Such methods can vary depending on the type of biomolecule measured. Methods for measuring RNA levels include, without limitation, PCR-based methods and in situ hybridization. For example, RT-PCR can be used with oligonucleotide primers designed to amplify CB and stefin A mRNA from a prostate cancer sample. Once amplified, the products corresponding to CB and stefin A mRNA can be separated by gel electrophoresis, and the levels of CB and stefin A product determined by densitometry. The levels determined by densitometry can be used to calculate a CB/stefin A ratio. The level of enzyme and inhibitor mRNA also can be measured in a sample by in situ hybridization. For example, prostate samples (e.g., whole prostates from prostatectomy patients or prostate biopsies) can be collected and frozen. Adjacent sections prepared from the frozen prostate samples can be hybridized with biotinylated oligonucleotide CB probes to localize CB mRNA and biotinylated oligonucleotide stefin A probes to localize stefin A mRNA using techniques similar to those reported elsewhere (Sinha et al., Anat. Rec., 235:233-240 (1993)). Other sections can be hybridized with a control probe (e.g., biotinylated pBR322). Hybridizations to localize CB and stefin A are typically performed under moderate to high stringency conditions. Moderate to high and high stringency conditions are well known in the art. For example, moderate stringency conditions can include those conditions provided herein, e.g., hybridizing at about 37° C. in a hybridization solution containing 4×SSC, 50% deionized formamide, 10% dextran sulphate, 1×Denhardt's solution, 0.025% yeast tRNA, 0.05% denatured salmon testis DNA, 1 mM ribonucleoside vanadyl complexes, 3 ng/ml oligodeoxythymidine-12 mers, and 10 to 15 ng/μl of biotinylated probes, while the washes are performed at about 37° C. with a wash solution containing 4×SSC and 50% formamide solution. High stringency conditions can include hybridizing at about 42° C. in a hybridization solution containing 25 mM KPO₄ (pH 7.4), 5×SSC, 5×Denhart's solution, 50 μg/mL denatured, sonicated salmon sperm DNA, 50% formamide, 10% Dextran sulfate, and 1-15 ng/mL probe (about 5×10⁷ cpm/μg), while the washes are performed at about 65° C. with a wash solution containing 0.2×SSC and 0.1% sodium dodecyl sulfate. Hybridization conditions can be adjusted to account for unique features of the nucleic acid molecule, including length and sequence composition. Probes can be labeled (e.g., fluorescently) to facilitate detection. The distribution of the CB and stefin A probes can be quantitated using an image analysis system. Prostate sections treated with RNAse before hybridization or incubated with a control probe can be used as controls to confirm that localization of CB and stefin A probes is specific. The levels of CB and stefin A mRNA quantitated by image analysis can be used to calculate a CB/stefin A ratio.

Methods for measuring polypeptide levels include, without limitation, ELISA-, immunohistochemistry-, and immunofluorescence-based techniques. CB and stefin A polypeptide levels in a prostate sample can, for example, be measured using a quantitative sandwich ELISA technique. Prostate tissue samples can be homogenized and extracted, and aliquots of the extracts added to separate wells of a microtiter plate pre-coated with antibodies specific for CB or stefin A. After protein binding and subsequent washing, enzyme-linked antibodies (e.g., mouse monoclonal antibodies) specific for CB or stefin A polypeptides can be added to the wells. After antibody binding and subsequent washing, a substrate solution containing a label-conjugated anti-mouse IgG can be added to the wells (e.g., horseradish peroxidase (HRP)-conjugated anti-mouse IgG). The label then can be quantitated by spectrophotometry and the quantitated levels used to determine a CB/stefin A ratio. Polypeptide levels of other enzymes and their inhibitors also can be measured in a sample using commercially available ELISA-based immunoassays such as the cathepsin D or MMP kits from Calbiochem (San Diego, Calif.).

To improve the accuracy of such assays, a starting material containing both malignant and non-malignant tissues can be enriched for the malignant portions. For example, cancerous glands within a prostate sample section can be individually collected by laser capture microdissection. The collected cancerous glands then can be homogenized, extracted, and processed by quantitative sandwich ELISA as described herein. Such a method provides a ratio that can more accurately reflect a malignant tumor's degree of aggressiveness.

Polypeptide levels also can be measured by immunohistochemistry. For example, a prostate sample section can be treated with anti-CB primary antibodies, while an adjacent section can be treated with anti-stefin A primary antibodies. Negative control sections can be incubated with pre-immune rabbit or mouse serum in lieu of the primary antibodies. After antibody binding and subsequent washing, the primary antibodies can be detected with appropriate label-conjugated secondary antibodies (e.g., gold-conjugated or enzyme-conjugated antibodies). The label is then developed and quantitated using an image analysis system. The resulting quantitated polypeptide levels can be used to calculate a CB/stefin A ratio.

Immunofluorescence techniques represent another approach to measuring the level of a polypeptide. For example, CB and stefin A polypeptides can be localized in the same prostate sample section using polyclonal or monoclonal antibodies against CB and stefin A. The bound antibodies are detected using different fluorescently-conjugated antibodies. The levels of CB and stefin A fluorescence are quantitated using an image analysis system, and the quantitated levels used to calculate a CB/stefin A ratio.

Suitable antibodies for ELISA-, immunohistochemistry- and immunofluorescence-based methods can be obtained using standard techniques. In addition, commercially available antibodies to enzymes and inhibitors involved in cancer invasion and metastasis such as those from DAKO USA (Carpinteria, Calif.), KRKA (Nova Mesto, Slovania), and Calbiochem can be used.

The invention provides kits that can be used to determine the levels of CB and stefin A biomolecules in a sample. Components and methods for producing kits are well known. Kits can contain oligonucleotide primers or probes for detecting CB and stefin A nucleic acids, or antibodies for detecting CB and stefin A polypeptides. CB and stefin A antibodies that are components of the kits provided herein typically have specific binding affinity for either CB or stefin A. “Specific binding affinity” as it relates to an antibody describes an antibody's ability to interact specifically with a particular polypeptide without significantly cross-reacting with other different polypeptides in the same environment. An antibody having specific binding affinity for CB can interact with CB polypeptides specifically in the presence of multiple different polypeptides, for example, multiple different cathepsins. The kits provided herein also can contain a reference chart that indicates a reference level for CB and stefin A polypeptides or RNAs. Kits can be configured in any type of design (e.g., microtiter plate design) and can be made of any type of material (e.g., plastic).

The invention will be further described in the following examples, which do not limit the scope of the invention described in the claims.

### EXAMPLES

### Example 1

Measuring a CB/Stefin A mRNA Ratio

Human prostate tissue pieces were collected immediately after surgery from 41 patients undergoing prostatectomy for adenocarcinoma of the prostate. These patients had not been treated with hormones or cytotoxic agents preoperatively. Data showing pre-surgery serum PSA (prostate specific antigen) levels and clinical stage of the disease were also collected. All specimens and data were collected according to the standard guidelines. Prostate tissue samples were processed as reported elsewhere (Sinha et al., Anat. Rec., 235:233-240 (1993)). Briefly, small pieces of prostate samples, frozen in liquid nitrogen or on dry ice, were stored at −70° C. until analysis. The remaining pieces were fixed in cold 3 to 4% paraformaldehyde (PF) in 0.01 M phosphate-buffered saline (PBS) or 0.1M phosphate buffer containing 2.5% sucrose and 5 mM magnesium chloride prepared in 0.02% diethylpyrocarbonate (DEPC)- treated and autoclaved double distilled water. All solutions were made in DEPC-treated and autoclaved double distilled water. Formalin-fixed samples were washed in cold PBS (several changes) to remove fixative and stored in 70% ethanol at 4° C. until paraffin and/or paraplast embedding and staining with hematoxylin and eosin (H&E). H&E stained cryostat and formalin fixed sections were used in histological grading of prostate cancer. Only cryostat sections of frozen samples were used in CB and stefin A mRNA localization by in situ hybridization techniques. Salient features of the techniques are described below.

Treatment of Cryostat Sections

Frozen specimens, embedded in OCT (Lab Tek Products, Miles Lab. Inc., Naperville, Ill.), were sectioned at 4 to 6 μm with a cryostat and placed on autoclaved, alcohol cleaned, 1% poly-L-lysine-coated slides. Selected unfixed cryostat sections were incubated with 20 μg/ml RNAse A in RNAse buffer (0.5 M NaCl, mM Tris-HCl, pH 8.0) for 30′ at 37° C. and rinsed thoroughly with RNAse buffer. RNAse A-treated and untreated cryostat sections were air dried and fixed in 100% cold acetone for 10′ and washed in cold PBS to remove OCT. Stefin A and CB mRNA probes were localized in RNAse-treated and untreated sections. A biotinylated oligonucleotide probe pBR322 (Synthetics Genetics, Inc., San Diego, Calif.) was used as a control probe.

Selection and Synthesis of Cathepsin B and Cystatin A mRNA Probes

An antisense 25-base nucleotide sequence oligonucleotide probe for human CB mRNA (GGAGGTGTCGGCGGAGGACCTGCTC; SEQ ID NO:1) corresponding to amino acids 50-58 according to Fong et al. (Proc. Natl. Acad. Sci. U.S.A., 83:2909-2913 (1986)) and a 27-base oligonucleotide sequence probe for human cystatin A mRNA (AAATTGGAAGCTGTGCAGTATAAAACT; SEQ ID NO:2) corresponding to amino acids 154-179 according to Kartasova et al. (Nuclei Acids Res., 15:5945-5962 (1987)) were synthesized at the University of Minnesota Microchemical Facility using the Biosearch Model 8750 Oligonucleotide Synthesizer (Multi Gen/Biosearch, Nevada, Calif.). Both probes were biotinylated and purified according to the Biosearch protocols. Melting points for these probes were calculated for 4×SSC (saline sodium citrate; 1×SSC=150 mM NaCl, 15 mM Na citrate, pH 7.2 ) and 50% formamide according to the empirical formula (Davis et al. (1986) Basic Methods in Molecular Biology. Elsevier Science, N.Y.). The melting point for the CB antisense probe was 47.6° C. with 18 GC bases. The melting point for the stefin A antisense probe was 34.7° C. with 9 GC bases.

In Situ Hybridization of CB and Cystatin A Probes

Using techniques described elsewhere (Sinha et al., Anat. Rec., 235:233-240 (1993)), cryostat sections were hybridized with biotinylated CB and cystatin A probes. Cryostat sections were wetted for 5′ with 25 to 50 μl of hybridization mixture (HM) without the probe. The HM contained 4×SSC, 50% deionized formamide, 10% dextran sulphate, 1×Denhardt's solution, 0.025% yeast tRNA, 0.05% denatured salmon testis DNA, 1 mM ribonucleoside vanadyl complexes, and 3 ng/ml oligodeoxythymidine-12 mers (Sigma Co., St. Louis, Mo.). Each section was hybridized with 25 to 50 μl of HM containing 10 to 15 ng/μl of biotinylated CB mRNA or cystatin A probes, covered with parafilm and incubated at 37° C. for 4 hours in humidified trays. All sections were washed with 4×SSC and 50% formamide solution (three changes at 37° C.). Hybridized sections were rinsed again with 2×SSC (two changes) for 30′ each at 37° C. and finally with 1×SSC for 15′ at room temperature. They were processed according to the Dako in situ hybridization kit and protocol as described elsewhere (Sinha et al., Anat. Rec., 235:233-240 (1993)).

Control Studies

Several control studies were performed and evaluated. To demonstrate that the endogenous RNA was essential for production of CB or stefin A reaction products after in situ hybridization, unfixed, cryostat sections were incubated with 20 μg/ml RNase A in a RNAse buffer for 30′ at 37° C. and processed to localize CB or stefin A probe. To demonstrate that the reaction products were specific and not due to unbound message, selected hybridized sections were post-treated with RNAse A (20 μg/ml) and examined for any changes in reaction products. To establish that reaction products were due to CB and stefin A probes, selected cryostat sections were treated with a biotinylated oligonucleotide control probe (pBR322) and examined for the reaction products.

Gleason Grading System

All prostate tumors were graded according to the Gleason grading system for histologic scores (Gleason, Hum. Pathol., 23: 273-279 (1992) and Gleason and Vacurg (1977) Histologic grading and clinical staging of prostatic carcinoma. In Urologic pathology: the prostate (Tannenbaum M, ed) Lea & Febiger, Philadelphia, Pa. 171-213). The Gleason histologic scores were recorded for each hybridized section and compared with H&E stained sections. The present study included neoplastic prostates from Gleason scores 3 to 10; no samples of Gleason score 2 and 8 tumors were available. Frozen prostate samples for low (3 and 4) and high (9 and 10) histological score tumors were grouped together. Prostate sections were graded by one or two lab personnel. Distribution of normal prostate, BPH, and neoplastic prostate is provided in Table 1.

Quantitative Morphometrics by Computer-Based Image Analysis

Reaction products in normal prostate, BPH, and malignant prostate were examined by a Zeiss Axioplan microscope and quantitated using a computer-based image analysis system as described elsewhere (Sinha et al., Anticancer Research, 18: 1385-1392 (1998)). Briefly, a Photometric digital camera (Photometrics, Tucson, Ariz.) attached to a Zeiss microscope collected images on an Intel-driven PC using Metamorph software (Universal Imaging Corp., West Chester, Pa.). The threshold mapping created boundaries of the sections on the basis of gray values ranging from 4095 to 0, white and black, respectively. All objects included within the designated gray value range were expressed as a percentage of the total field area under view. For the in situ hybridization study, the threshold included objects showing gray value in the range of the reaction products. Five to 18 different measurements were made for each tissue section studied. All images were acquired at 200×magnification.

Statistical Methods

The classification of the ratios was performed using a two-sample t test with p<0.05 considered significant. Differences between CB/stefin A ratio classification and serum PSA levels were assayed using ANOVA with a p<0.05 considered significant.

General Observations

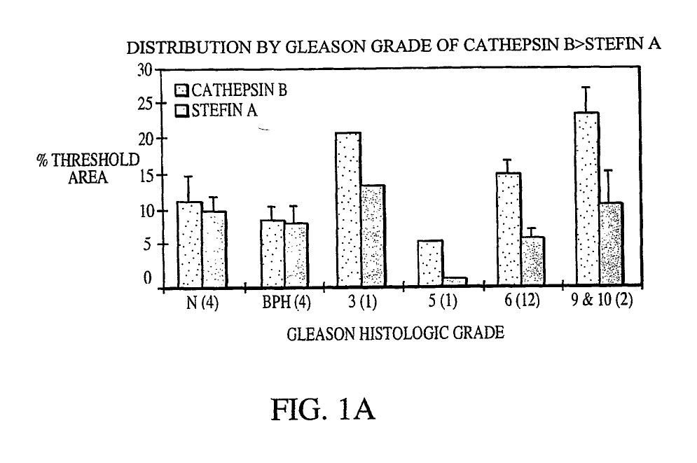

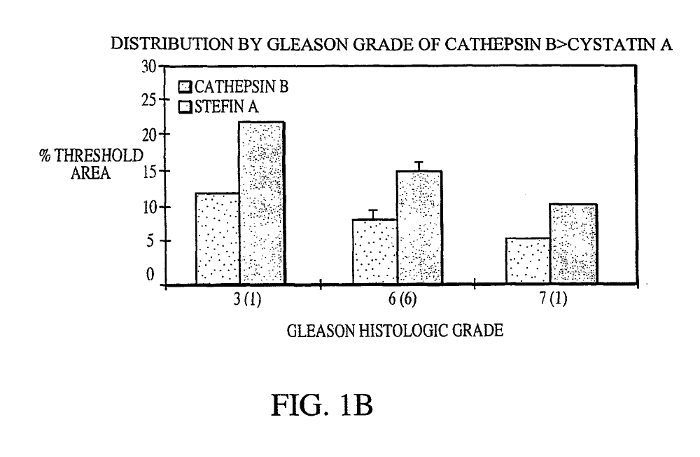

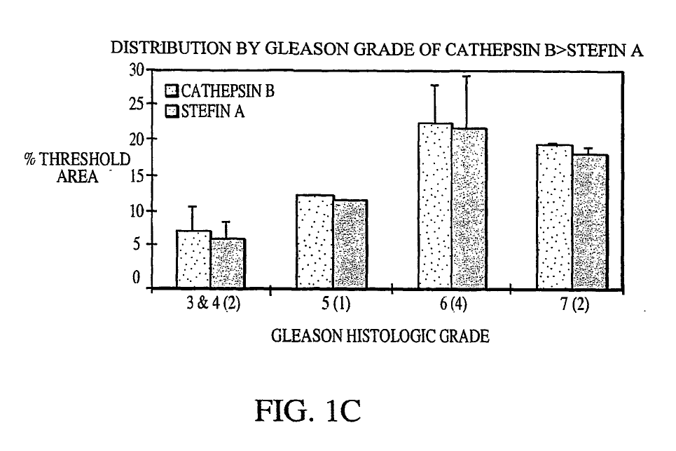

The in situ hybridization of cryostat sections revealed reaction products for CB and stefin A probes in normal, BPH and neoplastic prostates. There was little or no reaction product, however, in prostate sections pretreated with RNAse or hybridized with a control probe (pBR322). Post-treatment of hybridized sections with RNAse did not remove reaction product, suggesting that the localization was due to binding of the probes. Localization and distribution patterns of CB and stefin A probes were specific in prostate since these messages were absent in preRNAse-treated prostate sections. The analysis of localization data revealed that there were prostate cancer cases within Gleason histologic scores that expressed high, similar, and low levels of CB when compared to stefin A reaction products (Table 1 2nd FIGS. 1a, b, c). In Table 1, the distribution of the age of patients, clinical stage of the disease, pre-surgery levels of serum PSA, and the ratios of CB to stefin A in relation to the Gleason grading system are reported.

Cathepsin B and Stefin A Probes in Normal and BPH

Four normal and four BPH samples showed CB and stefin A mRNA reaction products predominantly in the cytoplasm of acinar basal cells and some columnar/cuboidal cells. Little or no CB or cystatin A reaction products were observed in stromal cells. In these prostates, reaction products for CB and stefin A mRNA were similar (FIG. 1a).

Cathepsin B and Stefin A Probes in Malignant Prostate

33 malignant prostates representing Gleason histologic scores 3 to 10 tumors were examined for localization of CB and stefin A mRNA. The reaction products were usually found in the cytoplasm of neoplastic acini and invasive cells and sometimes in stromal cells. Both morphologic and quantitative analysis of CB and stefin A mRNA reaction products showed considerable variations within and between Gleason scores. Most were Gleason score 6 tumors; other histologic scores were represented by relatively few samples (Table 1). Analysis of 22 Gleason score 6 tumors indicated that they could be divided into three patterns: neoplastic prostates showing higher levels of CB than stefin A, similar levels of CB and stefin A, and lower levels of CB than stefin A (FIGS. 1a, b, c). This relationship was also demonstrated by the ratios of CB to stefin A (Table 1). The Gleason score 6 tumors showed that the disease was represented by the B, C, and D clinical stages, with serum PSA levels ranging from 1 to 78 ng/ml. There was no correlation of the CB/stefin A ratio with the serum level of PSA in the 33 patients in this study. The remaining malignant prostates (Gleason scores 3, 4, 5, 7, 9, and 10 tumors) were also evaluated for these patterns and arranged along with the Gleason score 6 tumors (FIGS. 1a, b, c). Gleason score 3 and 4 tumors showed three patterns of localization and ratios for CB to stefin A (FIGS. 1a, b, c). Since the number of samples for scores 7, 9, and 10 were limited, only two patterns were identified (Table 1) (FIGS. 1a, b, c).

TABLE 1
Ratios of Cathepsin B and Stefin A in Human Prostate Cancer according to the Gleason Grading System.

| Gleason | Age at | | Pre-surgery | | | |
|---|---|---|---|---|---|---|
| Histologic Score | Prostatectomy | Clinical | serum PSA ng/ml | | | |
| (# of patients) | mean (range) | Stage | mean (range) | CB > Stef A | CB < Stef A | CB = Stef A |
| 3 & 4 (4) | 66 (63-68) | B, D | 49 (16-81) | 1.54 (25%) | 0.52 (25%) | 1.12 (50%) |
| 5 (2) | 67 (65, 67) | B | 9.6 (7, 12) | 5.41 (50%) | — | 1.03 (50%) |
| 6 (22) | 67 (56-87) | B, C, D | 12 (1-78) | 2.30 (55%) | 0.58 (27%) | 1.01 (18%) |
| 7 (3) | 58 (53-63) | B | 14 (4-34) | — | 0.59 (66%) | 1.09 (33%) |
| 9 & 10 (2) | 64 (61,67) | D | 78 (1, 156) | 2.26 (100%) | — | — |

The % threshold areas were used in determining the cut-off levels which were as follows: more than 1.5 for CB > stef A, less than 0.7 for CB < stef A; and 0.7-1.5 for CB = stef A. Parenthesis denotes percentage of patients showing GB to Stefin A ratios for each Gleason Histologic score. The total number of malignant prostates was 33.

Using several control studies (such as a control probe pBR322, pre-and-post-RNAse treatments of cryostat sections and stringency washes), the specificity of cDNA probes for CB and stefin A mRNA expression in normal prostate, BPH, and neoplastic prostate was demonstrated. In addition, it was shown that in some neoplastic prostates CB mRNA could be higher and lower than in normal prostate and BPH whereas it could be similar to that of the normal prostate in others. When studied independent of one another, CB and stefin A mRNA levels did not exhibit any specific relationship to the Gleason grading system. Calculating CB/stefin A mRNA ratios from levels measured in adjacent section did, however, reveal prostate cancers with different degrees of aggressiveness within the same Gleason score. Thus, CB and its inhibitors can be evaluated together for predicting prostate cancer progression.

Each Gleason score contained tumors with ratios of CB>stefin A, CB=stefin A, and CB<stefin A. Each Gleason score, therefore, contained tumors that varied in aggressiveness. Serum PSA levels did not correlate to CB/stefin A ratios or Gleason score (Table 1). Furthermore, serum PSA levels varied considerably within and between Gleason histologic scores and there was no correlation of CB/stefin A ratio and serum PSA. Since similar levels of CB and stefin A were found in BPH and normal prostate, it is reasonable to suggest that this is the normal relationship in prostates without malignancy. Therefore, the prostate cancer cases showing ratios of CB to stefin A similar to or less than that of the normal prostate and/or BPH can be considered variants of prostate cancer with less aggressive phenotypes for tumor progression than those exhibiting higher ratios of CB to stefin A (Table 1). These data demonstrate that the ratios of CB to stefin A mRNAs in prostate cancer samples can be used to define aggressive and less aggressive cancer phenotypes within a Gleason histological score.

### Example 2

Measuring a CB/Stefin A Polypeptide Ratio

Human prostatic tissue was collected from 45 patients immediately after prostatectomy for cancer or BPH, or cystectomy for bladder cancer. These individuals were not treated with hormones or cytotoxic agents preoperatively. All specimens were collected according to the standard guidelines. In addition, information on the age of the patient, clinical stage of the disease at prostatectomy, and pre-surgery serum total PSA (prostate specific antigen) level of each patient were collected. Small pieces of neoplastic and hyperplastic prostates were frozen in liquid nitrogen or dry ice and the remaining pieces were fixed in cold 3 to 4% paraformaldehyde (PF) in 0.01 M phosphate-buffered saline (PBS) or 0.1 M phosphate buffer containing 2.5% sucrose. Frozen samples were stored at −70° C. for the study. The PF-fixed samples were washed in cold PBS (several changes) to remove fixative and stored in 70% ethanol at 4° C. until paraffin and/or paraplast embedding and sectioning.

Prostate Sections

Frozen specimens were embedded in OCT (Lab Tek Products, Miles Lab. Inc., Naperville, Ill.), sectioned with a cryostat, placed on 1% poly-L-lysine coated slides, air dried, fixed in cold 100% acetone for 5 minutes, and used for hematoxylin and eosin (H&E) staining and immunofluorescence microscopy. Frozen prostate tissue sections were used for colocalization of CB and stefin A by confocal microscopy. Both cryostat and formalin-fixed sections were stained with H&E and used in pathological diagnosis. The H&E-stained sections were compared with those examined by phase contrast microscopy prior to their examination by epifluorescence and confocal microscopy.

Antibodies Against CB and Stefin A

Polyclonal rabbit antihuman CB (PC 41) was obtained from Oncogene Research Products (Calbiochem, Cambridge, Mass.). Monoclonal mouse anti-human stefin A antibody was obtained from KRKA, (Nova Mesto, Slovenia). Donkey anti-mouse-Cy3 (rhodamine) and donkey anti-rabbit-FITC (fluorescein) were purchased from Jackson ImmunoResearch (West Grove, Pa.). Phosphate buffered saline and bovine serum albumin (BSA) were obtained from Sigma Chemical Co. (St. Louis, Mo.). Cathepsin B and stefin A antibodies in this study were affinity purified as described by the manufacturers.

Localization of CB and Stefin A by Immunofluorescence and Confocal Microscopy

In a preliminary study, the optimal conditions for stefin A and CB protein localization in the same prostate tissue sections were determined. For localization of CB and stefin A in the same section, cryostat sections were fixed in 100% acetone, blocked with 2% BSA (30 minutes), incubated overnight at 4° C. with mouse anti-human stefin A (1:10), washed with PBS, and incubated for 1 hour at 37° C. with donkey anti-mouse-Cy3 (1:500). Sections were washed again with PBS and blocked for 30 minutes with 2% BSA before incubation for 1 hour at 37° C. with rabbit anti-human CB (1:50). The sections were washed and incubated with donkey anti-rabbit-FITC (1:100) for 1 hour at 37° C. Sections were washed with PBS and mounted with aqua mount (Polysciences Inc., Warrington, Pa.). The mounted sections were examined for CB (FITC) and stefin A (Cy3) localization using a Zeiss epifluorescence microscope or for CB/stefin A colocalization using a confocal microscope (Bio-Rad MRC-1000 or MRC-1024 confocal microscope equipped with a krypton/argon laser, Bio-Rad Life Sciences Group, Hercules, Calif.). Final processing of images was done with Adobe Photoshop (San Jose, Calif.). Colocalization techniques described elsewhere were followed (Sinha et al., Anat. Rec. 252:281-289 (1998)).

Negative Control Studies

In negative control experiments for immunofluorescence and confocal microscopic study, prostate sections were treated with normal mouse immunoglobulin (IgG) and washed in PBS. The same section was treated by normal rabbit serum and washed in PBS and incubated with Cy3 and FITC labeled secondary antibodies. These sections were washed with PBS and mounted with aqua mount for the study.

Gleason Grading System

All prostate tumors were graded according to the Gleason grading system that is based primarily on the degree of architectural glandular differentiation and predominant histologic patterns. Tumor heterogeneity in the Gleason system was accounted for by assigning a primary pattern of dominant grade and a secondary pattern of the non-dominant grade; histological scores were obtained by adding these two patterns together. Gleason histologic scores were determined using H&E-stained slides prepared from frozen and formalin-fixed specimens and compared with those used in phase contrast microscopy and immunostaining. Although this study focuses on Gleason score 6 tumors, prostate tumors showing other histologic scores (2 to 10) were included. Prostate sections were graded for pathological diagnosis by lab personnel. Since frozen prostate tissue samples for Gleason score 2 to 4 and 8 to 10 were limited in number, 2-4 tumors were grouped together as well-differentiated and 8-10 as poorly-differentiated prostate cancers. Moderately-differentiated tumors are represented by scores 5 to 7, but score 6 was the focus of this study.

Quantification of Data by Metamorph Image Analysis System

Localization of FITC and Cy3 tagged molecules was entered directly from the microscope slides to a computer-based image analysis system. Briefly, images collected from Bio-Rad scanning confocal microscope were analyzed using Metamorph software (Universal Imaging Corp., West Chester, Pa.). The threshold mapping of the fluorescence in tissue sections created boundaries on the basis of gray values ranging from 4095 to 0, white and black, respectively. All objects included within the designated gray value range were expressed as a percentage of the total field area under view at the magnification of 200×. The threshold localization included objects showing gray value in the range of fluorescence and 3 to 6 measurements were made for each localization study. All images were acquired at a magnification of 200. Statistical significance was determined using Student's t test at p<0.05.

Structure of Prostate Glands and Their Tumors

The structure of normal prostate, BPH, and neoplastic prostate is described elsewhere (Sinha et al., Anat. Rec. 223: 266-275 (1989); Sinha et al., The Prostate, 26:171-178 (1995), and Sinha et al., Anat. Rec. 235: 233-240 (1993)). Briefly, human prostate contains numerous glandular acini and prostatic ducts. Prostatic acini usually contain basal, neuroendocrine, and cuboidal and/or columnar cells and are often separated by variable amounts of connective tissues containing muscle fibers, fibroblasts, macrophages, blood vessels, lymphatics, and nerve fibers. In neoplastic prostates, cancerous glands and invasive cells vary in size, shape, distribution, and neoplastic cells in the invasive edges were often indicative of their progression in prostatic stroma.

General Patterns of CB and Stefin A Localization

Analysis of prostate cancer patients by age, clinical stage, total serum PSA level, and Gleason score showed that the Gleason score provided the most consistent relationship with cellular distribution of CB and stefin A (Table 2). Since PSA is not involved in degradation of extracellular matrix proteins, the above parameters were arranged according to the Gleason scores (Table 2). In addition, serum PSA levels and clinical stages varied within and between Gleason scores. No specific relationship between serum PSA levels and the ratios of CB to stefin A was found. Hematoxylin and eosin-stained section (for light microscopy) and unstained sections (for phase contrast microscopy) showed the structural details of prostate glands and connective tissue whereas immunofluorescence and confocal microscopy illustrated co-distribution of CB and stefin A in neoplastic prostate and BPH glands. CB was localized by fluorescein (FITC) and stefin A by rhodamine (Cy3). Colocalization produced yellow fluorescence. The negative control sections did not show FITC for CB, Cy3 for stefin A, or yellow fluorescence by confocal microscopy.

TABLE 2
Ratios of Cathepsin B and Stefin A within Gleason Histologic Scores of Human Prostate Cancer.

| Gleason | Age at | | Pre-surgery | | | |
|---|---|---|---|---|---|---|
| score | prostatectomy | Clinical | serum PSA ng/ml | | | |
| (# of patients) | mean(range) | stage | mean(range) | CB = stefin | CB > stefin A | GB < stefin A |
| 2-4 (5) | 73 (68-77) | B | 7.2 (1.5-16) | 1.18 (60%) | 3.41 (40%) | — |
| 5 (1) | 66 | B | 3.7 | — | — | 0.52 (100%) |
| 6 (18) | 68 (62-76) | B, C | 9.3 (3-22) | 0.95 (45%) | 5.57 (22%) | 0.37 (33%) |
| 7 (6) | 61 (55-64) | C, D | 28 (19-34) | — | 2.61 (33%) | 0.51 (66%) |
| 8-10 (3) | 64 (62-69) | C, D | 56 (1-157) | 1.12 (33%) | 7.36 (66%) | — |
| BPH (12) | | | | 0.99 (100%) | | |

The % threshold areas of CB = stefin A in BPH were used as a baseline for the cut-off levels for neoplastic prostates which were as follows: CB = stefin A, 0.7 to 1.5; CB > stefin A, more than 1.5; CB < stefin A, less than 0.7. The number of patients examined for each ratio is in parenthesis. The ratio of GB = stefin A was not significantly different from the ratio seen in BPH. The ratios of CB > stefin A and CB < stefin A were significantly
# higher or lower when compared to BPH or CB = stefin A in neoplastic prostates (p < 0.05). Heterogeneity in Gleason score 6 tumors is shown by the ratios of CB = stefin A, CB > stefin A, and CB < stefin A. Heterogeneity in the other Gleason score groups was also seen, but to a lesser extent due to low sample sizes. The total number of malignant prostates was 33 and total number of BPH prostates was 12.

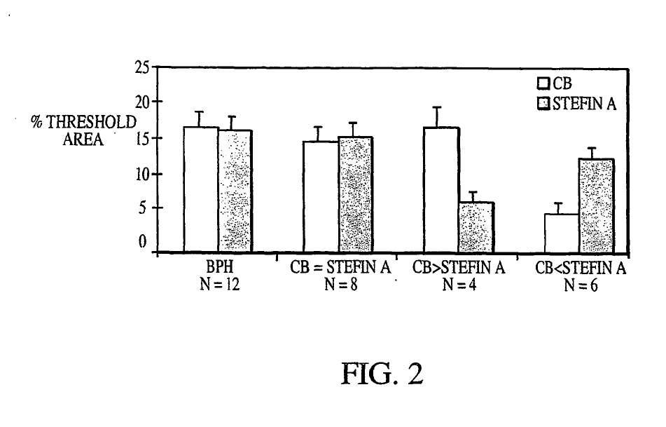

Twelve BPH samples were studied by immunofluorescence and/or confocal microscopy. CB and stefin A proteins localized predominantly in basal cells and many cuboidal/columnar cells of BPH glands as shown by FITC and Cy3 fluorescence, and also by yellow fluorescence of FITC and Cy3 colocalization in the same cells. Colocalization of CB and stefin A indicated that many sites for CB and stefin A in BPH glandular cells overlapped. Stromal connective tissue contained occasional macrophages showing yellow fluorescence. Inage analysis of CB and stefin A protein localization in BPH glands showed that the levels of CB to stefin A polypeptides were equal (Table 2; FIG. 2), similar to the situation with CB and stefin A mRNAs.

Neoplastic Prostate

Thirty-three neoplastic prostates were examined by immunofluorescence and/or confocal microscopy. Localization of CB or stefin A polypeptides individually in neoplastic prostate glands, including individual or groups of invasive cells in prostatic stroma, showed variations in fluorescence within Gleason score 6 tumors. This was also observed in other tumors with different Gleason scores (Table 2). When analyzed independently of one another, CB and stefin A polypeptide levels did not show any specific relation to Gleason scores, clinical stages, or pre-prostatectomy PSA levels. CB/stefin A polypeptide ratios, however, exhibited a relationship with Gleason scores. For example, Gleason score 6 tumors showed high, moderate, and low levels of CB and stefin A by colocalization. In addition, the ratios of CB to stefin A and colocalization data could be arranged according to their distribution patterns, namely CB=stefin A, CB>stefin A, and CB<stefin A. When the ratios of CB to stefin A were CB>stefin A and CB<stefin A, there were fewer sites of yellow fluorescence than when the ratio was CB=stefin A. This data also showed that the ratio of FITC (CB) and Cy3 (stefin A) fluorescence for CB=stefin A was about 1, as was found in BPH. In contrast, the ratios of FITC and Cy3 fluorescence for CB>stefin A and CB<stefin A were significantly higher and lower than 1, respectively (Table 2). Since the other Gleason score tumors were represented by relatively few prostate samples, it was found that the three patterns were not present in the same score (Table 2). Further analysis of CB and stefin A indicated that both the enzyme and inhibitor existed in the same cell, but usually at different sites in neoplastic prostate. Occasional macrophages showed FITC, Cy3, or yellow fluorescence.

The data show that the cellular distributions of CB and stefin A polypeptides and the ratios of CB to stefin A polypeptides (e. g., CB=stefin A, CB>stefin A and CB<stefin A) in prostate cancers with Gleason histologic score 6 were similar to the distributions and ratios seen with CB and stefin A mRNA. The ratio of CB to stefin A polypeptides was about 1 in BPH. Because BPH is a non-malignant condition, it may be that prostate tumors (malignant or non-malignant) have a homeostatic relationship between enzyme and inhibitor. In other words, CB enzyme may not be available for extracellular matrix degradation and remodeling in tumors with CB/stefin A ratios close to 1. Tumors with CB/stefin A ratios greater than 1 exhibit a more aggressive phenotype than tumors with CB/stefin A ratios equal to or less than 1 because they have higher levels of CB enzyme needed for degradation of extracellular matrix proteins and progression of tumor cells from prostatic acini to stromal compartments in the primary organ. Further, because prostate cancers have high levels of mature CB compared to its proenzyme form, an excess of enzyme over inhibitor would facilitate degradation of extracellular matrix proteins. The regulation of CB function in cancer cells, in addition to concentration of enzyme and inhibitor, is affected by subcellular localization. Thus, the most aggressive prostate cancer cells may be those cells containing the mature form of the enzyme in the presence of a low concentration of inhibitor. These data demonstrate that prostate cancers have high levels of mature CB enzyme and a heterogeneous ratio of enzyme to inhibitor polypeptide. Tumors showing CB<stefin A ratios can be less aggressive because there is more inhibitor than the enzyme, thus disfavoring degradation of extracellular matrix proteins and tumor cell progression. The data also demonstrate heterogeneity within a single Gleason score category when using CB/stefin A polypeptide ratios.

### Example 3

Plasma Membrane Association of Cathepsin B in Human Prostate Cancer: Biochemical and Immunogold Electron Microscopic Analysis

Human prostatic tissue was collected from 29 patients immediately after prostatectomy for cancer or BPH, or cystectomy for bladder cancer. These individuals were not treated with hormones or cytotoxic agents preoperatively. All specimens were collected according to the standard guidelines. Small pieces of benign (‘normal’), neoplastic, and hyperplastic prostates were frozen in liquid nitrogen or dry ice and the remaining pieces were fixed in cold 3 to 4% paraformaldehyde (PF) in 0.01 M phosphate-buffered saline (PBS) or 0.1M phosphate buffer containing 2.5% sucrose. Frozen samples were stored at −70° C. for the study. The PF-fixed samples were washed in cold PBS (several changes) to remove fixative and stored in 70% ethanol at 4° C. until paraffin, paraplast, or K4M resin (Polysciences Inc., Warrington, Pa.) embedding and sectioning.

Prostate Sections

Frozen prostate specimens were embedded in OCT (Lab Tek Products, Miles Lab. Inc., Naperville, Ill.), sectioned with a cryostat, placed on 1% poly-L-lysine coated slides, fixed in cold 100% acetone for 5 minutes and stained by hematoxylin and eosin (H & E) staining. Frozen prostate tissue samples were used for homogenization and measurement of CB activities in the tissue extracts. Fresh prostate tissues were used for isolation of lysosomal and plasma membrane fractions, and for measuring CB and cysteine protease inhibitor (CPIs) activities (see below). Pellets of lysosomes and membrane/endosomal fractions were prepared by centrifugation, fixed in paraformaldehyde, embedded in K4M resin and thin sectioned by an Ultratome (see below). Phosphate buffered saline and bovine serum albumin (BSA) were obtained from Sigma Chemical Co.(St. Louis, Mo.).

Gleason Grading System

All prostate tumors were graded according to the Gleason grading system. Diagnosis of prostate cancer, normal prostate, and BPH was made on frozen and fixed H&E-stained sections by a surgical pathologist, who also graded resin-embedded prostate sections according to the Gleason grading system. This study is based upon 15 BPH and 14 prostate cancer cases. In this study, the prostate tissue samples included Gleason histologic score 5 to 7 tumors.

Tissue Homogenization and Subcellular Fractionation

Frozen prostate tissue pieces (1-5 mm thick) from 18 prostates were embedded in OCT (Lab Tek Products, Miles Lab., Inc., Naperville, Ill.), sectioned at 6 μm, and stained with H&E for determining Gleason scores. Cancerous areas were grossly dissected while frozen from adjacent BPH and benign prostate areas. Tissue pieces that were at least 70% cancerous on histologic examination were used as cancer specimens for this study as were BPH tissue pieces with no histological evidence of malignancy. For homogenization and determination of CB activities in extracts, frozen specimens of 10 BPH and 8 prostate cancers were used. Samples were thawed, washed three times in 0.01 M PBS to remove OCT, blotted, weighed. A 10% homogenate was prepared by disruption of minced tissue in 0.1% Triton X-100 (pH 6.5) using 3 bursts of 15 seconds each with a Polytron (Brinkmann, Littau, Switzerland) at setting of 5. The homogenates were centrifuged at 960×g for 10 minutes and the supernatant was saved as the extract and frozen at −70° C.

Subcellular fractionation studies were conducted on 3 BPH and 2 neoplastic prostate tissues following the techniques described elsewhere (Rozhin et al., Cancer Res. 47:6620 (1987)). Briefly, all manipulations of homogenates and subcellular fractions were done at 4° C. Fresh prostate tissue pieces were minced in ice-cold homogenization buffer (250 mM sucrose, 25 mM 2-[N-morpholino] ethanesulfonic acid (Sigma, St. Louis, Mo.), 1 mM EDTA, pH 6.5) and homogenized with a Polytron. The homogenate was filtered through four layers of cheesecloth, and the filtrate volume was adjusted to 10% (w/v) with homogenization buffer. The initial homogenate was separated into four fractions, namely, nuclear and heavy mitochondrial fraction, light mitochondrial (L) fraction, lysosomal (L-1) and plasma membrane/endosomal fractions (L-2). Fractions enriched in plasma membrane and endosomal vesicles were isolated by sequential differential and 30% isoosmotic Percoll (Sigma) density gradient centrifugation. Because purified CB is inactivated above pH 7, the Percoll fractionations were performed at neutral pH. Fractions were collected in one of two ways: either two visible bands (L-1 and L-2) were aspirated with a Pasteur pipette; or 0.4 ml fractions were collected from the bottom of the tube using a peristaltic pump. In the latter case the homogenization buffer used was 250 mM sucrose, 25 mM N-(2-hydroxyethyl) piperazine-N′-2-ethanesulfonic acid,−1 mM EDTA, pH 7.3. Fractions were re-suspended in or diluted with homogenization buffer, and frozen at −70° C.

Assay of Cathepsin B and Cysteine Protease Inhibitor Activity

CB activities were assayed in the extracts of the frozen BPH and cancerous tissues using Z-Arg-Arg-4-methoxy-β-napthylamide (Z-Arg-Arg-MNA) (Bachem. Inc., Torrence Calif.) as substrate and the methods described by Barrett and Kirschke (Methods Enzymol. 80:535-561 (1981)). The arg-arg sequence confers considerable specificity to CB as compared with other cathepsins (Mort et al., Biochem. J. 233:57-63 (1986)). A 20 mM stock solution of substrate was prepared using 5% dimethylformamide. The final reaction volume was 0.40 ml containing 75 mM phosphate buffer (pH 6. 0), 1 mM EDTA, 2 mM cysteine and 0.5 mM Z-Arg-Arg-MNA. The reaction was started by addition of tissue extracts and terminated after incubation at 40° C. for 10 minutes by addition of 0.40 ml coupling reagent. After completion of color development, 1.0 ml butan-1-ol was added per tube. The tubes were vortexed vigorously for 15 seconds, the organic phase was clarified by centrifugation and the optical density of the organic phase determined at 520 nm. The activity is expressed as nmoles of 2-naphthylamine released as determined from a standard curve. CB activities were measured in the cell fractionation studies using a more sensitive fluorometric assay with carbobenzyloxyarginylarginyl-7-amido-4-methylcoumarin (Z-Arg-Arg-NHMec) as substrate. The activities of CB in homogenates and in membrane/endosomal fractions derived from them were determined according to published protocols (Rozhin et al., Biochem. Biophys. Res. Comm. 164:556-561 (1989) and Sloane et al, Proc. Nat. Acad. Sci. U.S.A. 83:2483-2487 (1986)). CB activities were determined as Vₘₐₓ to minimize interactions with endogenous competitive inhibitors.

The total activity of heat stable CPIs against the plant cysteine protease papain was assayed according to our published procedure (Rozhin et al., Biochem. Biophys. Res. Comm. 164:556-561 (1989)). For this purpose, homogenates and isolated cellular fractions were heated to 100° C. for 5 minutes. This process releases the inhibitors and inactivates CB. Activity of the heat stable inhibitors is expressed as inhibitory units with one unit representing the amount of inhibitory protein that will completely inhibit the release by papain of one μmole of product/minute.

Other Assays

Protein was determined using the bicinchoninic acid method of Pierce (Micro BCA assay, Pierce Chemical Co., Rockford, Ill.) using BSA as standard. DNA was measured by the method of Burton (Biochem J. 62:315-323 (1956)).

Antibodies

Purified, monospecific, rabbit polyclonal antibody immunoglobulin against human liver CB (as described by Moin et al., Biochem J. 285:427-434 (1992)), and another affinity purified, polyclonal antibody against human liver CB obtained from Oncogene Research Products (Calbiochem, Cambridge, Mass.) were used. Immunohistochemical localization of CB was similar with the two antibodies (Sinha et al., Anat. Rec. 223: 266-275 (1989) and Sinha et al., The Prostate, 26:171-178 (1995)). In addition, an affinity purified rabbit anti-PSA IgG from Dako Corp. Inc. (Carpinteria, Calif.) and normal rabbit serum obtained from Sigma was used. Goat anti-rabbit IgG conjugated with 10 to 15 nm gold particles were obtained from Jensen, Inc. (SPI supplies, PA) and Sigma.

Localization of CB by Immunogold Electron Microscopy

Five BPH and 6 prostate cancer samples were studied of which 3 BPH and 2 prostate cancer specimens were used in the subcellular fractionation studies and an additional 2 BPH and 4 prostate cancer cases were studied by immunogold electron microscopy. CB was localized by immunogold electron microscopic techniques as described by elsewhere (Sinha et al., Anat. Rec. 223: 266-275 (1989) and Sinha et al., The Prostate, 26:171-178 (1995)). Briefly, pellets were prepared by centrifugation at 120,000×g, fixed in 3 to 4% paraformaldehyde in 0.1 M phosphate buffer, embedded in K4M resin, and thin sectioned by an Ultratome. Thin sections of pellets and prostate samples were incubated overnight at 4° C. with the primary antibody, rabbit anti-human liver cathepsin B, washed in PBS and treated with goat anti-rabbit IgG conjugated with 10 to 15 nm conjugated gold particles. Sections were stained by lead citrate and uranyl acetate, washed in PBS and examined by a Zeiss 10 C electron microscope.

Control Studies

In negative control studies, prostate sections were incubated with diluted normal rabbit serum and goat anti-rabbit IgG conjugated gold particles and processed for localization study. For another control study, rabbit anti-PSA was localized in lieu of the primary antibody against CB followed by treatment with goat anti-rabbit IgG conjugated gold particles

Structure of Prostate Glands and their Tumors

Human prostate contains numerous glandular acini and prostatic ducts. Prostatic acini usually contain basal, neuroendocrine, and cuboidal and/or columnar cells and are often separated by variable amounts of connective tissues containing muscle fibers, fibroblasts, macrophages, blood vessels, lymphatics, and nerve fibers. In neoplastic prostates, cancerous glands and invasive cells vary in size, shape, distribution, and invasive edges that are often indicative of tumor cell progression in prostatic stroma (Sinha et al., The Prostate, 26:171-178 (1995)).

Biochemical Activities of CB in BPH and Cancerous Prostate

Extracts of frozen prostate tissues that were predominantly cancer or BPH were used for biochemical measurements of CB activities using a chromogenic peptide substrate. The relative amounts of protein or DNA were not significantly different in the homogenates of hyperplastic or moderately-differentiated (Gleason scores 5 to 7) tumor tissues. Furthermore, there were no differences in activity of CB in neoplastic and hyperplastic prostate whether the activity was expressed per mg protein or per mg DNA (Table 3).

TABLE 3
CB activities in the extracts of BPH and prostate cancer tissues.

| | | | | μmoles Z-arg-arg- | |
|---|---|---|---|---|---|
| | | | | OMen A/10 min | |
| | | mg Protein | mg DNA | per mg | |
| Tissue | Number | g Tissue | g Tissue | protein | per mg DNA |
| BPH | 10 | 35.8 ± 2.4* | 1.3 ± 0.3 | 0.8 ± 0.1 | 25.3 ± 2.6 |
| CAP** | 8 | 33.7 ± 6.4 | 1.3 ± 0.5 | 0.9 ± 0.1 | 22.8 ± 2.7 |

*Mean ± S.E.M. CAP, prostate cancer; BPH, benign prostatic hyperplasia
**The extracts of CAP tissue assayed for CB activity were from 3 patients with Gleason score 5 and 5 patients with Gleason score 7.

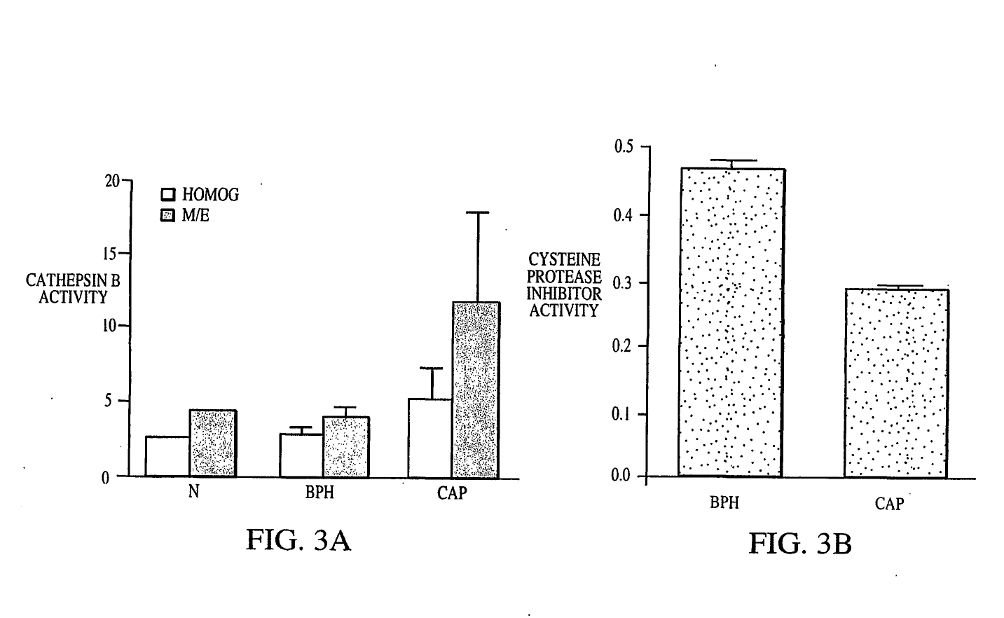

The activity of CB was also measured with a fluorogenic assay in homogenates of fresh prostate tissues and in subcellular fractions derived from them. In these assays, multiple substrate concentrations were used to determine the Vₘₐₓ of CB activity because the presence of endogenous CPIs can affect the accurate quantitation of CB activities in homogenates. CB activities determined with the fluorogenic peptide substrate did not differ significantly in homogenates of BPH and neoplastic prostate (FIG. 3a). This result was the same found with the chromogenic peptide substrate. CB activities were, however, more variable in plasma membrane/endosomal fractions of neoplastic prostate than BPH (FIG. 3a). CB activities in the membrane/endosomal fractions of prostate cancer were significantly higher than in BPH (FIG. 3a). In other words, CB was more closely associated with the plasma membrane/endosomal fractions of neoplastic prostates than those of BPH (FIG. 3a).

When tissues are disrupted during homogenization for subcellular fractionation, the binding of CPIs to CB can prevent accurate quantification of the cysteine protease activities. The activity of total CPIs can be assessed by taking advantage of the heat stability of the CPIs and heat lability of the cysteine proteases, such as CB. The total heat stable CPI activity was about 40% lower in homogenates and plasma membrane/endosomal fractions of neoplastic prostate than in those of hyperplastic prostate (FIG. 3b). In other words, the activity of heat stable CPIs was significantly higher in BPH than in prostate cancer (FIG. 3b). Further analysis of the data revealed that the CB to CPI ratio was 8±0 in homogenate and 25±1 in plasma membrane/endosomal fractions of BPH glands, whereas the CB to CPI ratio was 26±1 in homogenate and 117±2 in plasma membrane/endosomal fractions of prostate cancer. These data demonstrate a shift in balance of enzyme to inhibitor favoring CB activity in prostate cancer. In addition, the specific activity of CB is greater than that of CPI in the plasma membrane/endosomal fraction versus homogenate, indicating that CB and its inhibitors are in different subcellular compartments. Thus, the capacity to regulate cysteine protease activities through inhibitors in prostate cancers may be reduced when compared to non-malignant prostat tissues.

Localization of CB in Lysosomal and Membrane/Endosomal Fractions

The structural profiles of lysosomal and plasma membrane/endosomal fractions in BPH and neoplastic prostates were analyzed. In thin sections, profiles of lysosomes appeared as light and dense vesicles of variable densities, but only lysosomes showed localization of CB by immunogold electron microscopy. Very small components in plasma membrane/endosomal fractions were unidentifiable morphologically, but localization of CB in them suggested that they were fragments of plasma membranes. In addition, occasional light and dense vesicles showed localization of gold particles at the outer surface of these vesicles suggesting that gold particles adhered to plasma membrane/endosomal fragments. Prostate glands produce and secrete PSA, which is found in numerous secretory vesicles and granules. PSA was localized to the lysosomal fractions to determine if secretory granules were present in them. Localization of PSA by immunogold electron microscopy in occasional secretory granules and vesicles was found. The absence of PSA localization in dense vesicles in the preparations confirmed that vesicles localizing CB did not contain PSA. The negative control study of lysosome and plasma membrane/endosomal fractions and absence of PSA in lysosomes demonstrated the specificity of CB localization by immunogold electron microscopy.

Localization of CB in BPH and Neoplastic Prostate Tissue Samples

Thin sections of prostate tissues from which the pellets of lysosomal and membrane/endosomal fractions were obtained and prostates not used in fractionation studies showed specific localization of CB by immunogold particles. In BPH glands, lysosomes were often present in the perinuclear cytoplasm of the glandular cells. In contrast, CB in malignant prostates localized in many lysosomes that were adjacent to the luminal and/or basal surfaces of cancerous glands and invasive cells. In the invasive cells, which do not have basal or luminal surfaces, lysosomes were present near the outer surfaces. In cancer cells, occasional perinuclear lysosomes were also observed by immunogold electron microscopy.

Although CB activities were not increased in homogenates of prostate cancer versus those of hyperplastic prostate, there was a significant increase in CB activities in plasma membrane/endosomal fractions of malignant prostates. Concurrently, there were decreased activities of heat stable cysteine protease inhibitors in neoplastic prostate when compared to BPH. These findings indicate that there was a shift in the distribution of CB from one of a predominantly lysosomal localization in BPH glands to one that includes the plasma membrane/endosomal fraction in malignant prostate. Analysis of CB and CPIs indicated that the plasma membrane/endosomal compartment of malignant prostate had a low level of CPI activity. The decline in CPI activity probably occurs due to a shift in control of CB activity that would favor distribution of the enzyme to the apical and basal cell surfaces in prostate cancer.

The analysis of lysosomal and plasma membrane/endosomal fractions revealed that lysosomes in BPH localized CB whereas the enzyme was predominantly associated with the plasma membrane fractions of malignant prostate. Since the pellets did not show structural relationships of cytoplasmic and nuclear constituents, the distribution of CB in thin sections of tissues was determined by immunogold electron microscopy. Lysosomes containing CB were distributed predominantly in the perinuclear areas of the cytoplasm in BPH, while they were near the plasma membranes (basal and luminal surfaces) of invasive cells and neoplastic prostate glands. The observed shift in CB localization to the cell surfaces in malignant prostate but not in BPH is consistent with the earlier observations that CB is localized to the inner and/or outer surfaces of tumor cells of many human tumors.

The distribution of CB is distinctively different in epithelial cells of normal organs and non-malignant tumors when compared to malignant tumors. Thus, the altered distribution of CB and its increased activities can be treated as specific characteristics of malignant human tumors, including prostate cancer.

The increased activities of CB are associated with the plasma membrane/endosomal preparation along with the diminished activity of its inhibitor activity in malignant prostates. Immunogold microscopy has confirmed that there is an altered distribution of lysosomes containing CB from the perinuclear cytoplasm to that near the plasma membrane in malignant prostates. Thus, the altered distribution of CB and lysosomes can be used in differentiation of malignant versus non-malignant prostates.

### Example 4

### Predicting Lymph Node Metastases with CB/Stefin A Ratios Measured in Primary Organ Tumors

Human prostate tissue samples were collected from 74 prostate cancer patients and 8 BPH patients immediately after prostatectomy. The patients were not treated with hormones or cytotoxic agents preoperatively. All samples were collected according to standard guidelines. Prostate tissue samples were fixed in neutral buffered formalin and then embedded in paraffin or paraplast. Once embedded, 4 to 6 μm sections were cut from the samples. One section from each sample was stained with H&E, and the adjacent sections were used for other analyses. The stained sections were graded according to the Gleason histologic score grading system. Fourteen stained sections representing separate prostate samples received Gleason scores of 2, 3, 4, 5, 9, or 10. Those samples were removed from further study. The remaining 60 stained sections representing separate prostate samples received Gleason scores of 6, 7, or 8.

Some of the adjacent sections representing the 60 remaining prostate cancer samples and the 8 BPH samples were processed for immunofluorescence. Briefly, the sections were treated with 2% BSA to block non-specific antibody binding. After 30 minutes, the blocked sections were treated with mouse anti-human stefin A antibodies (KRKA) overnight at 4° C. The sections were washed with PBS, and then treated with donkey anti-mouse-Cy3 (Jackson ImmunoResearch). After 1 hour at 37° C., the sections were washed again with PBS and blocked with 2% BSA for 30 minutes. The blocked sections were treated with rabbit anti-human CB (PC41; Oncogene Research Products) for 1 hour at 37° C. The sections were then washed with PBS, and then treated with donkey anti-rabbit-FITC (Jackson ImmunoResearch). After 1 hour at 37° C. the sections were washed with PBS and mounted on slides with aqua mount (Polysciences Inc.). The mounted sections were examined for CB and stefin A localization using a Zeiss epifluorescence microscope or for CB/stefin A colocalization using a confocal microscope (Bio-Rad MRC-1000 or MRC-1024 confocal microscope equipped with a krypton/argon laser; Bio-Rad Life Sciences Group, Hercules, Calif.)

Epiflorescence images of CB and stefin A localization were collected at a magnification of 200×. The collected images were analyzed using Metamorph software (Universal Imaging Corp.). Briefly, the images were processed using a threshold algorithm that mapped fluorescence boundaries (i.e., objects) within a section based on grayscale pixel values ranging from 4095 (white) to 0 (black). The threshold included objects having grayscale pixel values in the range of the fluorescence objects. The total area of all objects included within the designated gray value range was expressed as a percentage of the total field area under view.

Serum total PSA levels prior to prostatectomy ranged between 0.2 and 146 ng/ml (Table 4). The distribution of tumor positive and negative pelvic lymph nodes showed that Gleason score 6 and 8 tumor patients had fewer prostate tumor positive lymph nodes than Gleason score 7 tumor patients (Table 4). In addition, Gleason score 6 tumor patients had a greater proportion of prostate tumor negative lymph nodes tumors compared to Gleason score 7 and 8 tumor patients (Table 4). Regression analysis did not reveal any specific relationship between the ratios of CB to stefin A and Gleason score 6 tumors (p=0.6096, p=0.7632, respectively), Gleason score 7 and 8 tumors (data not shown), clinical stages (p=0.4156, 0.9795, respectively), PSA levels (p=0.4252, p=0.6050, respectively), and tumor positive pelvic lymph nodes (p=0.1236, p=0.9318, respectively).

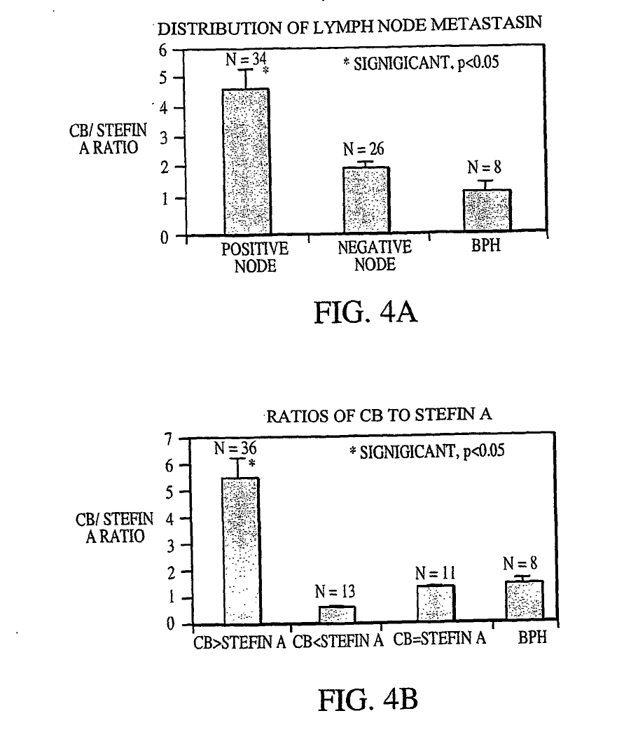

The fluorescence distribution of CB and stefin A in prostate tissue sections showed that mean CB levels were consistently higher than mean stefin A levels within a particular Gleason score. Both CB and stefin A mean values were not significantly different across the Gleason score 6 to 8 tumors (Table 4). Sections from Gleason score 6, 7, and 8 tumor samples showed three distinct ratios: CB>stefin A, CB<stefin A, and CB=stefin A. Samples that exhibited CB>stefin A ratios had significantly (p<0.05) higher incidence of pelvic lymph node metastasis than those with the ratios of CB<stefin A and CB=stefin A (FIGS. 4a, b). BPH samples exhibited a low incidence of pelvic lymph node metastasis (FIG. 4a). In addition, patients with CB>stefin A as a group exhibited a significant (p<0.05) increase in mortality compared to those with ratios of CB<stefin A and CB=stefin A (Table 5). These data demonstrate that CB/stefin A ratios measured in primary organ tumors can be used to predict lymph node metastasis in patients with prostate cancer. Further, these data demonstrate that increased CB/stefin A ratios correlate with increased mortality.

TABLE 4
Ratios of CB and Stefin A within Gleason Histologic Scores of Human Prostate
Cancer correlated with Lymph Node Metastasis and Mortality.

| Patient Profile | Gleason Score 6 | Gleason Score 7 | Gleason Score 8 | |
|---|---|---|---|---|
| Total # of samples | 28 | 23 | 9 | |
| Age | | | | |
| Mean ± SEM | 66.4 ± 0.6 | 64.0 ± 1.0 | 63.8 ± 1.9 | |
| (range) | (54-75) | (55-71) | (52-69) | |
| Clinical Stage | | | | |
| TNM range | T(1-4), N(1-3), M(0) | T(1-4), N(1-3), M(0-1) | T(2-4), N(1-3), M(0-1) | |
| Pre-surgery PSA | | | | |
| mean ± SEM | 7.1 ± 1.2 | 23.3 ± 4.8 | 70.5 ± 17 | |
| (range) | (1-25) | (0.2-44) | (30-146) | |
| | | | | Total |
| Lymph node + | 7 | 20 | 7 | 34 |
| (% within score) | (25%) | 87% | 75% | |
| Lymph node − | 21 | 3 | 2 | 26 |
| (% within score) | (75%) | (13%) | (22%) | |
| Mortality | 8 | 12 | 7 | 27 |
| (% within score) | (29%) | (52%) | (78%) | |
| Cathepsin B | | | | |
| mean ± SEM | 5.93 ± 1.1 | 4.95 ± 1.1 | 5.12 ± 1.2 | |
| (range) | (0.4-26) | (0.2-23) | (0.8-11) | |
| Stefin A | | | | |
| mean ± SEM | 2.41± 0.5 | 3.08 ± 0.6 | 2.36 ± 0.7 | |
| (range) | (0.4-11) | (0.6-12) | (0.2-6) | |
| CB > Stefin A | | | | |
| mean ± SEM | 6.34 ± 2.2 | 4.06 ± 0.8 | 6.62 ± 2.3 | |
| (n) | 16 | 13 | 6 | |
| CB < Stefin A* | | | | |
| mean ± SEM | 1.03 ± 0.1 | 0.65 ± 0.1 | 0.81 ± 0.2 | |
| (n) | 12 | 10 | 3 | |

*comprised of CB < stefin A and CB = stefin A data.

### Example 5

Measuring a CB/Stefin A Polypeptide Ratio in Laser Capture Microdissected (LCM) Prostate Samples

Sections from four Gleason score 6 samples (described in Example 4) were processed for PSA immunohistochemistry. The PSA stained sections were then viewed by microscopy and photographed at a magnification of 200×. The resulting photographs established the PSA staining pattern in the sections prior to LCM. The stained sections were covered with transparent 100 μm-thick ethylene-vinyl acetate films. PSA-positive regions (representing cancerous glands) were then dissected from separate sections under a high power objective using a LCM machine (Arcturus Engineering Inc., Mountain View, Calif.) according to protocols described by Simone et al. (Am. J. Pathol., 156:445-452 (2000)). The dissected cancerous glands were extracted using a technique described by Ikeda et al. (J. Histochem. Cytochem., 46: 397-403 (1998)), and the resulting extracts were used to measure CB and stefin A polypeptide levels by ELISA. Briefly, separate extracted samples were added to separate wells of a microtiter plate pre-coated with monoclonal antibodies specific for CB (KRKA) or stefin A (KRKA). After 24 hours at 4° C., the wells were washed several times with 5% BSA in PBS. Enzyme-linked polyclonal rabbit antibodies specific for CB (Oncogene) or stefin A (KRKA) were then added to the wells. After 60 minutes at room temperature, the wells were washed several times with 0.05% Tween 20 in PBS. Following the washes, a solution containing horseradish peroxidase (HRP)-conjugated anti-mouse IgG was added to the wells. After 60 minutes at room temperature, the substrate TMB (3′, 3′, 5′, 5′ tetramethyl benzidine) was added. The TMB reaction was stopped by adding 50 μl 2 M H₂SO₄, and the intensity of the TMB color was measured at 450 nm using a microplate reader (Model 450, Bio-Rad, USA).

All four samples exhibited detectable levels of both CB and stefin A. Three of the four samples showed CB/stefin A ratios greater than 1.5 (Table 5). These three ratios were determined to be significant at 0.05 using the Student's t test. One sample was found to have a CB/stefin ratio less than 1.5. These data demonstrate that the combination of LCM and ELISA can be used to obtain CB/stefin A ratios from cancerous prostate tissue.

TABLE 5
CB/stefin A ratios obtained by LCM ELISA assay

| Sample ID | CB (ng/mL) | Stefin A (ng/mL) | CE/stefin A ratio |
|---|---|---|---|
| 80-01 | 79.8 | 15.0 | 5.31* |
| 85-39 | 65.0 | 25.2 | 2.58* |
| 86-17 | 25.7 | 24.0 | 1.07 |
| 87-29 | 50.0 | 15.9 | 3.13* |

*determined by Student's t test to be significant at p < 0.05.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for determining the aggressiveness of a cancer in a mammal, said method comprising determining the ratio of a cathepsin mRNA to a stefin mRNA in said mammal, and determining said aggressiveness based on said ratio.

2. The method of claim 1, wherein said cancer is determined to be aggressive if said ratio is greater than 1.0.

3. The method of claim 1, wherein said cancer is determined to be aggressive if said ratio is greater than 3.0.

4. The method of claim 1, wherein said cancer is determined to be aggressive if said ratio is greater than 5.0.

5. The method of claim 1, wherein said cancer is selected from the group consisting of prostate, breast, skin, brain, colon, lung, ovary, and bladder cancers.

6. The method of claim 1, wherein said cancer is prostate cancer.

7. The method of claim 1, wherein said mammal is a human.

8. The method of claim 1, wherein said ratio is determined in tissue from said cancer.

9. The method of claim 1, wherein said ratio is determined by RT-PCR or in situ hybridization.

10. The method of claim 1, wherein said cathepsin mRNA is a CB mRNA.

11. The method of claim 1, wherein said stefin mRNA is a stefin A mRNA.

12. A method for determining the aggressiveness of a cancer in a mammal, said method comprising determining the ratio of cathepsin polypeptide to stefin polypeptide in said mammal, and determining said aggressiveness based on said ratio.

13. The method of claim 12, wherein said cancer is determined to be aggressive if said ratio is greater than 1.0.

14. The method of claim 12, wherein said cancer is determined to be aggressive if said ratio is greater than 3.0.

15. The method of claim 12, wherein said cancer is determined to be aggressive if said ratio is greater than 5.0.

16. The method of claim 12, wherein said cancer is selected from the group consisting of prostate, breast, skin, brain, colon, lung, ovary, and bladder cancers.

17. The method of claim 12, wherein said cancer is prostate cancer.

18. The method of claim 12, wherein said mammal is a human.

19. The method of claim 12, wherein said ratio is determined in tissue from said cancer.

20. The method of claim 12, wherein said ratio is determined by ELISA, confocal microscopy, immunofluorescence, or immunohistochemistry.

21. The method of claim 12, wherein said cathepsin polypeptide is a CB polypeptide.

22. The method of claim 12, wherein said stefin polypeptide is a stefin A polypeptide.

23. A method for determining whether or not a cancer in a mammal is metastatic, said method comprising determining the ratio of a cathepsin polypeptide to a stefin polypeptide in said mammal, and determining whether or not said cancer is metastatic based on said ratio.

24. The method of claim 23, wherein said cancer is determined to be metastatic if said ratio is greater than 2.5.

25. The method of claim 23, wherein said cancer is determined to be metastatic if said ratio is greater than 3.0.

26. The method of claim 23, wherein said cancer is determined to be metastatic if said ratio is greater than 5.0.

27. The method of claim 23, wherein said cancer is determined to be metastatic if said ratio is greater than 7.0.

28. The method of claim 23, wherein said cancer is selected from the group consisting of prostate, breast, skin, brain, colon, lung, ovary, and bladder cancers.

29. The method of claim 23, wherein said cancer is prostate cancer.

30. The method of claim 23, wherein said mammal is a human.

31. The method of claim 23, wherein said ratio is determined in tissue from said cancer.

32. The method of claim 23, wherein said ratio is determined by ELISA, confocal microscopy, immunofluorescence, or immunohistochemistry.

33. The method of claim 23, wherein said cathepsin polypeptide is a CB polypeptide.

34. The method of claim 23, wherein said stefin polypeptide is a stefin A polypeptide.

35. A kit comprising a first oligonucleotide primer pair and a second oligonucleotide primer pair, wherein said first oligonucleotide primer pair amplifies a CB nucleic acid and said second oligonucleotide primer pair amplifies a stefin A nucleic acid.

36. A kit comprising a first oligonucleotide probe and a second oligonucleotide probe, wherein said first oligonucleotide probe hybridizes under moderate to high stringency conditions to a CB nucleic acid and said second oligonucleotide probe hybridizes under moderate to high stringency conditions to a stefin A nucleic acid.

37. The kit of claim 36, wherein said probes are labeled.

38. A kit comprising a first antibody and a second antibody, wherein said first antibody has specific binding affinity for a CB polypeptide and said second antibody has specific binding affinity for a stefin A polypeptide.
